(19) United States
(12) Patent Application Publication
(10) Pub. No.: US 2023/0285307 A1
(43) Pub. Date: Sep. 14, 2023

(54) Dispersible Tablet Formulations Comprising Dolutegravir

(71) Applicant: ViiV Healthcare Company, Wilmington, DE (US)

(72) Inventors: Ian Paul CONN, Ware, Hertfordshire (GB); Mark Robert DAVIES, Ware, Hertfordshire (GB); Joanne HEAFIELD, Ware, Hertfordshire (GB); Michael HOLTON, Ware, Hertfordshire (GB); Neil MORTIMER, Ware, Hertfordshire (GB)

(73) Assignee: ViiV Healthcare Company, Wilmington, DE (US)

(21) Appl. No.: 18/001,635

(22) PCT Filed: Jun. 23, 2021
(86) PCT No.: PCT/IB2021/055533

§ 371 Date: Dec. 13, 2022

(30) Foreign Application Priority Data
Jun. 25, 2020 (GB) 2009684.8

(51) Int. Cl. A61K 9/20; A61K 9/28; A61K 31/5383

(52) U.S. Cl. CPC A61K 9/2077; A61K 9/2018; A61K 9/28; A61K 31/5383

(57) ABSTRACT

The present invention relates to formulations comprising dolutegravir or a pharmaceutically acceptable salt thereof, processes for making such formulations, and the use of such formulations in the treatment of HIV infection, in particular in the treatment of HIV infection in pediatric patients.

### FIELD OF THE INVENTION

The present invention relates to formulations comprising dolutegravir or a pharmaceutically acceptable salt thereof, processes for making such formulations, and the use of such formulations in the treatment of HIV infection, in particular in the treatment of HIV infection in pediatric patients.

### BACKGROUND TO THE INVENTION

Human immunodeficiency virus (“HIV”) infection and related diseases are a major public health problem worldwide. Human immunodeficiency virus type 1 (“HIV-1”) is a retrovirus which encodes three enzymes that are required for viral replication: reverse transcriptase, protease, and integrase. Drugs targeting reverse transcriptase, protease and integrase are in wide use and have shown effectiveness, particularly when employed in combination.

HIV infection however remains a major medical problem, with tens of millions of people still infected worldwide. The World Health Organization reported that 2.6 million children of less than 15 years of age were living with HIV-1 in 2014. Although therapeutic options for this patient population have improved, additional pediatric formulations of antiretroviral agents are needed.

Dolutegravir is an integrase strand transfer inhibitor (INSTI). Dolutegravir inhibits HIV integrase by binding to the integrase active site and blocking the strand transfer step of retroviral DNA integration which is essential for the HIV replication cycle.

The chemical name of dolutegravir is (4RX,12aS)-N-[(2,4-difluorophenyl)methyl]-7-hydroxy-4-methyl-6,8-dioxo-3,4,12,12a-tetrahydro-2H-pyrido[5,6]pyrazino[2,6-b][1,3]oxazine-9-carboxamide (CAS Registry Number 1051375-16-6). Dolutegravir has the following structural formula:

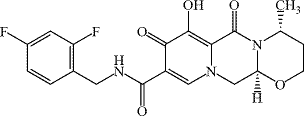 [SMILES: C[C@@H]1CCO[C@H]2Cn3cc(C(=O)NCc4ccc(F)cc4F)c(=O)c(O)c3C(=O)N12]

Dolutegravir sodium (TIVICAY^{®}) is approved for use in a broad population of HIV-infected patients. It is recommended for first-line treatment of HIV-1 infected adults due to it potency, high barrier to resistance and tolerability. It is also approved for children and adolescents aged 6 to 18 years of age.

The adult dosage form of TIVICAY^{®} is a tablet comprising 50 mg (free acid equivalent) of dolutegravir sodium. However, some patients, in particular pediatric patients, have difficulties swallowing tablets and generally require a different oral drug delivery system. There remains a need for alternative formulations of dolutegravir or a pharmaceutically acceptable salt thereof which are suitable for use in the treatment of HIV infection in certain patients, in particular pediatric patients.

### SUMMARY OF THE INVENTION

In a first aspect, the present invention provides a dispersible tablet formulation comprising dolutegravir or a pharmaceutically acceptable salt thereof and a pharmaceutically acceptable ion.

In a second aspect, the present invention provides a process for making a dispersible tablet formulation comprising dolutegravir or a pharmaceutically acceptable salt thereof and a pharmaceutically acceptable ion comprising mixing dolutegravir or a pharmaceutically acceptable salt thereof and a pharmaceutically acceptable ion.

In a third aspect, the present invention provides a dispersible tablet formulation comprising dolutegravir or a pharmaceutically acceptable salt thereof and a pharmaceutically acceptable ion for use in therapy.

In a fourth aspect, the present invention provides a dispersible tablet formulation comprising dolutegravir or a pharmaceutically acceptable salt thereof and a pharmaceutically acceptable ion for use in the treatment of HIV infection, in particular HIV infection in a pediatric patient.

In a fifth aspect, the present invention provides a method of treating HIV infection, for example HIV infection in a pediatric patient, which method comprises administering to said patient a dispersible tablet formulation comprising dolutegravir or a pharmaceutically acceptable salt thereof and a pharmaceutically acceptable ion.

In a sixth aspect, the present invention provides a kit comprising a dispersible tablet formulation comprising dolutegravir or a pharmaceutically acceptable salt thereof and a pharmaceutically acceptable ion, together with instructions for the use thereof for the treatment of HIV infection.

In a seventh aspect, the present invention provides a combination of a dispersible tablet formulation comprising dolutegravir or a pharmaceutically acceptable salt thereof and a pharmaceutically acceptable ion, together with another therapeutic agent.

### DESCRIPTION OF DRAWINGS/FIGURES

FIG. 1 shows the solubility of dolutegravir (in mg/ml after 30 minutes) in types of potable water.

FIG. 2 is a flow diagram of the manufacturing process for preparing dolutegravir sodium granules following a high shear wet granulation process. The dolutegravir is mixed with intragranular excipients followed by the controlled addition of water to allow the nucleation, a deagglomerating wet milling process, a fluid bed drying process to dryout the granules and a dry comilling step to provide the final granule size product. The granule batch is blended with extragranular components including calcium sulfate dihydrate and a portion used to manufacture the tablet formulation.

FIG. 3 shows that there is no difference in exposure in adults when 5×5 mg tablets are given as a dispersion compared with 5×5 mg tablets given direct to mouth.

### DETAILED DESCRIPTION OF THE INVENTION

As used herein, the term “comprise” and variations thereof, such as “comprises” or “comprising”, are to be construed in an open, inclusive sense, that is as “including, but not limited to”.

As used herein, the term “dispersible tablet formulation” refers to a tablet which disperses in aqueous phase, for example water, before administration or a tablet which disperses after administration direct to mouth. Typically, dispersible tablets are solid pharmaceutical forms which are defined by their rate of disintegration in water and the uniformity of dispersion of the particles into which they disintegrate. Dispersible tablet formulations may also be referred to as “tablets for oral suspension”.

The formulations of the present invention may be used to treat all patients including pediatric patients, adolescent patients and adult patients. In one embodiment, the formulations of the present invention are used to treat pediatric patients. As used herein, the term “pediatric patient” refers to a child of from 0 to 12 years of age, for example 4 weeks to 6 months, 6 months to 2 years or 2 years to 6 years.

As used herein, the term “pharmaceutically acceptable” with respect to a substance refers to that substance which is generally regarded as safe and suitable for use without undue toxicity, irritation, allergic response, and the like, commensurate with a reasonable benefit/risk ratio. “Pharmaceutically acceptable” with regard to excipients includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

As used herein, the term “pharmaceutically acceptable ion” refers to an ion which is added as a solubility modifier to reduce the solubility of dolutegravir in water. Suitable ions include, but are not limited to, sodium, potassium, magnesium and calcium. In one embodiment, the pharmaceutically acceptable ion is calcium.

Thus, in one embodiment, the present invention provides the use of a pharmaceutically acceptable ion as a solubility modifier in a dispersible tablet formulation comprising dolutegravir to reduce the solubility of dolutegravir in water. Suitable ions include, but are not limited to, sodium, potassium, magnesium and calcium. In one embodiment, the pharmaceutically acceptable ion is calcium.

“Pharmaceutically acceptable salt” refers to a salt of a compound that is pharmaceutically acceptable and that possesses (or can be converted to a form that possesses) the desired pharmacological activity of the parent compound. Such salts include, but are not limited to, acid addition salts formed with inorganic acids such as hydrochloric acid, hydrobromic acid, sulfuric acid, nitric acid, phosphoric acid, and the like; or formed with organic acids such as acetic acid, benzene sulfonic acid, benzoic acid, camphorsulfonic acid, citric acid, ethane sulfonic acid, fumaric acid, glucoheptonic acid, gluconic acid, lactic acid, maleic acid, malonic acid, mandelic acid, methane sulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quatemized ammonium salts. Representative non-limiting lists of pharmaceutically acceptable salts can be found in S.M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, PA, (2005), at p. 732, Table 38-5, both of which are hereby incorporated by reference herein.

As used herein, the term “salts” includes co-crystals. The term “co-crystal” refers to a crystalline compound comprising two or more molecular components, e.g. wherein proton transfer between the molecular components is partial or incomplete.

As used herein, the term “solvate” means a molecular complex comprising a compound and one or more pharmaceutically acceptable solvent molecules. Examples of solvent molecules include, but are not limited to, water and C₁₋₆ alcohols, e.g. ethanol. When the solvate is water, the term “hydrate” may be used.

As used herein, the term “%w/w” means the weight of a component as a percentage of the total weight of eg the granule or tablet core in which the component is present.

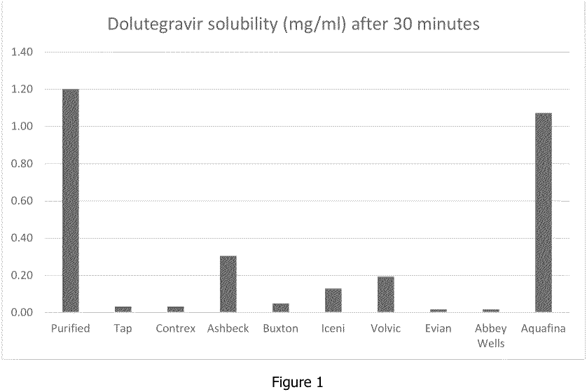

The solubility of dolutegravir is affected by the use of different types of potable water, for example purified water, tap water and different brands of bottled water (see FIG. 1). The reason for this difference in solubility is believed to be due to the presence of ions typically found in water. When water with no ions present (purified water) is used to disperse dolutegravir, the solubility of dolutegravir is increased and a significant bitter taste may be observed.

The bitter taste of dolutegravir may be significantly reduced when water comprising a suitable pharmaceutically acceptable ion is used for dispersion. As the water used by patients to disperse the tablets will vary, according to the present invention a pharmaceutically acceptable ion is incorporated into a dispersible tablet formulation in order to ensure the presence of a suitable pharmaceutically acceptable ion. The dispersible tablet formulation according to the invention has improved palatability, and thus increased compliance.

In a first aspect, the present invention provides a dispersible tablet formulation comprising dolutegravir or a pharmaceutically acceptable salt thereof and a pharmaceutically acceptable ion.

In one embodiment, the dolutegravir or a pharmaceutically acceptable salt thereof is dolutegravir sodium.

In another embodiment, the tablet comprises about 5 mg of dolutegravir (free acid equivalent).

The pharmaceutically acceptable ion is an ion which is added as a solubility modifier to reduce the solubility of dolutegravir in water. Suitable ions include, but are not limited to, sodium, potassium, magnesium and calcium. In one embodiment, the pharmaceutically acceptable ion is calcium.

The pharmaceutically acceptable ion will typically be incorporated into the dispersible tablet formulation in the form of a pharmaceutically acceptable salt, for example a sulfate salt such as calcium sulfate. The salts may be in anhydrous form or a hydate, for example a monohydrate or a dihydrate. In another embodiment, the calcium sulfate is calcium sulfate dihydrate.

In another embodiment, the pharmaceutically acceptable ion is present in an amount of up to about 5% w/w in the tablet core. In a another embodiment, the pharmaceutically acceptable ion is present in an amount of about 1 to about 4% w/w in the tablet core. In a another embodiment, the pharmaceutically acceptable ion is present in an amount of about 1.8 to about 2.6% w/w in the tablet core. In a further embodiment, the pharmaceutically acceptable ion is present in an amount of about 2.2% w/w in the tablet core.

Tablets of the invention will typically comprise one or more excipients. Excipients should be compatible with the other ingredients of the formulation and physiologically innocuous to the recipient thereof. Examples of suitable excipients are well known to the person skilled in the art of tablet formulation and may be found in, inter alia, “Handbook of Pharmaceutical Excipients”, 7^{th} Ed, 2012. As used herein the term “excipients” is intended to refer to, inter alia, processing aids, basifying agents, solubilisers, glidants, diluents (also known as bulking agents or fillers), binders, lubricants, surface active agents, disintegrants and the like. The term also includes agents such as sweetening agents, flavouring agents, colouring agents, preserving agents and coating agents. Such excipients will generally be present in admixture within the tablet.

Examples of processing aids include, but are not limited to, microcrystalline cellulose and silicified microcrystalline cellulose. The amount of processing aid in a tablet is generally from about 45 to about 65% w/w in the tablet core.

Examples of solubilisers include, but are not limited to, ionic surfactants (including both ionic and nonionic surfactants) such as sodium lauryl sulphate, cetyltrimethylammonium bromide, polysorbates (such as polysorbate 20 or 80), poloxamers (such as poloxamer 188 or 207), and macrogols.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet is generally from about 0.5 to about 2% w/w in the tablet core. In one embodiment, the lubricant is sodium stearyl fumarate.

Examples of disintegrants include, but are not limited to, starches, celluloses, cross-linked PVP (crospovidone) (Type A or Type B), sodium starch glycolate (Type A or Type B), croscarmellose sodium, etc. In one embodiment, the disintegrant is sodium starch glycolate (Type A). In a further embodiment, the disintegrant is crospovidone (Type B). The amount of disintegrant in a tablet is generally from about 9 to about 14% w/w in the tablet core.

Examples of diluents (also known as bulking agents or fillers) include, but are not limited to, starches, maltodextrins, polyols (such as lactose), and celluloses. For example, the diluent may be selected from mannitol, microcrystalline cellulose, silicified microcrystalline cellulose and lactose monohydrate.

In one embodiment, the diluent is mannitol. The amount of diluent in a tablet is generally from about 13 to about 19 % w/w in the tablet core.

Examples of binders include, but are not limited to, cross-linked PVP, HPMC, sucrose, starches, etc. In one embodiment, the binder is a povidone. In a further embodiment, the binder is povidone K29/32. The amount of binder in a tablet is generally from about 1 to about 2% w/w in the tablet core.

Examples of sweetening agents include, but are not limited to, sucralose, sucrose, saccharin and polyols (such as mannitol and sorbitol). In one embodiment, the sweetening agent is sucralose.

In one embodiment, the present invention provides a dispersible tablet comprising dolutegravir or a pharmaceutically acceptable salt thereof, a pharmaceutically acceptable ion and a sweetening agent.

In another embodiment, the sweetening agent is present in an amount of up to about 2% w/w in the tablet core. In another embodiment, the sweetening agent is present in an amount of about 0.1% to to about 1% w/w in the tablet core. In a further embodiment, the sweetening agent is present in an amount of about 0.6 to abut 0.8% w/w in the tablet core.

Examples of flavouring agents include, but are not limited to, strawberry, orange, banana, raspberry, peach, passion fruit, golden syrup or mixtures thereof. Flavours are readily available from commercial sources such as flavour houses, or can be developed by those skilled in the art. It will be appreciated that preferred flavours assist in masking the taste of the active ingredient(s) of the formulation. In one embodiment, the flavouring agent is strawberry. Representative strawberry flavours may comprise natural flavours, synthetic equivalents thereof, or artificial flavours or mixtures thereof. In another embodiment, the strawberry flavour is strawberry cream flavour, for example PHS-132963 available from the flavour house Givaudan.

In one embodiment, the present invention provides a dispersible tablet formulation comprising dolutegravir or a pharmaceutically acceptable salt thereof, a pharmaceutically acceptable ion and a flavouring agent. In another embodiment, the present invention provides a dispersible tablet formulation comprising dolutegravir or a pharmaceutically acceptable salt thereof, calcium sulfate and strawberry cream flavour.

In another embodiment, the flavouring agent is present in an amount of up to about 2% w/w in the tablet core. In another embodiment, the flavouring agent is present in an amount of about 0.1% to to about 1% w/w in the tablet core. In a further embodiment, the flavouring agent is present in an amount of about 0.2 to about 0.4% w/w in the tablet core.

In one embodiment, the present invention provides a dispersible tablet formulation comprising dolutegravir or a pharmaceutically acceptable salt thereof, a pharmaceutically acceptable ion, a sweetening agent and a flavouring agent.

In one embodiment, tablets provided herein are uncoated. In a further embodiment, tablets provided herein are coated. Although uncoated tablets may be used, it is more usual in the clinical setting to provide a coated tablet, in which case a conventional coating may be used.

Film coatings are known in the art. They can be composed of hydrophilic polymer materials and include, but are not limited to, polysaccharide materials, such as hydroxypropyl methylcellulose (HPMC), methylcellulose, hydroxyethyl cellulose (HEC), hydroxypropyl cellulose (HPC), poly(vinylalcohol-co-ethylene glycol) and other water soluble polymers. Though in one embodiment the water soluble material included in the film coating of the embodiments disclosed herein includes a single polymer material, in certain other embodiments it is formed using a mixture of more than one polymer. In one embodiment, the coating is white.

Suitable coatings include, but are not limited to, polymeric film coatings such as those comprising polyvinyl alcohol e.g. Aquarius BP18237 White film coat or Opadry OY-S-28876 white film coat. The amount of coating is generally from about 2 to about 4% w/w of the tablet core. In one embodiment, the coating is about 3% w/w of the tablet core.

In a second aspect, the present invention provides a process for making a dispersible tablet formulation comprising dolutegravir or a pharmaceutically acceptable salt thereof and a pharmaceutically acceptable ion comprising mixing dolutegravir or a pharmaceutically acceptable salt thereof and a pharmaceutically acceptable ion.

In one embodiment, granules of dolutegravir or a pharmaceutically acceptable salt thereof are first separately prepared prior to blending with extragranular components including a pharmaceutically acceptable ion.

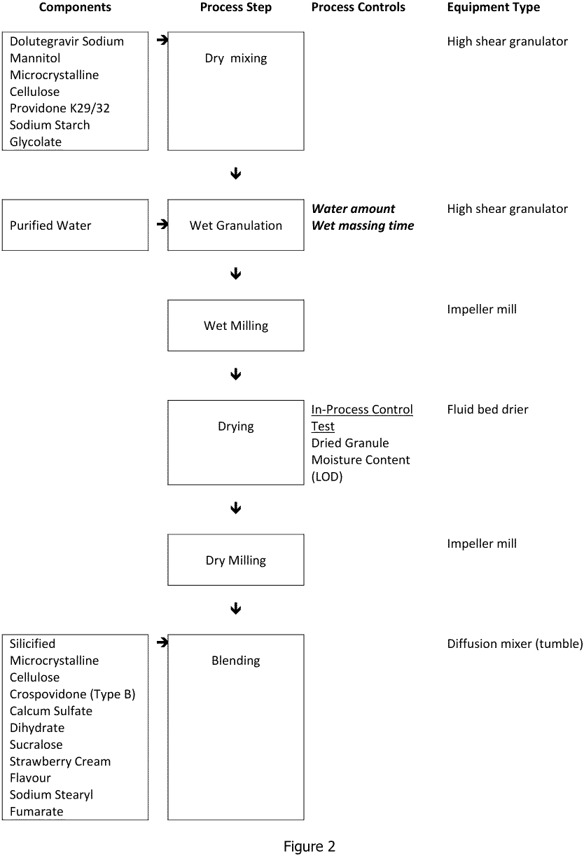

To make the dolutegravir granules, the active is first mixed with one or more of the aforementioned excipients in a suitable blender to blend the materials. In one embodiment, dolutegravir (as dolutegravir sodium) is admixed with a first amount of excipients by high shear granulation. This mixture is wet granulated and wet milled and the granules are then dried and then dry milled. Thereafter, a second amount of excipients are added to the granules and further blended. The final dolutegravir granules are collected in a suitable container. The flow diagram for dolutegravir sodium granule manufacture and subsequent blending is shown in FIG. 2.

In one embodiment, the dolutegravir sodium granules comprise dolutegravir sodium, a diluent, a processing aid, a binder and a disintegrant. In another embodiment, the dolutegravir granules comprise dolutegravir sodium, mannitol, microcrystalline cellulose, povidone and sodium starch glycolate.

In one embodiment, the dolutegravir sodium granules are blended with a processing aid, a disintegrant, calcium sulfate, a sweetener, a flavouring agent and a lubricant. In another embodiment, the dolutegravir sodium granules are blended with silicified microcrystalline cellulose, crospovidone, calcium sulfate dihydrate, sucralose, strawberry cream flavour and sodium stearyl fumarate.

In one embodiment, the dispersible tablet formulation of the invention comprises about 5 mg free acid equivalent of dolutegravir sodium, a pharmaceutically acceptable ion in an amount of about 1.8 to about 2.6% w/w in the tablet core, a diluent in an amount of from about 13 to about 19 % w/w in the tablet core, a processing aid in an amount of about 45 to about 65% w/w in the tablet core, a binder in an amount of about 1 to about 2% w/w in the tablet core and a disintegrant in an amount of about 9 to about 14% w/w in the tablet core.

In another embodiment, the dispersible tablet formulation of the invention comprises about 5 mg free acid equivalent of dolutegravir sodium, a pharmaceutically acceptable ion in an amount of about 1.8 to about 2.6% w/w in the tablet core, a sweetening agent in an amount of about 0.6 to abut 0.8% w/w in the tablet core and flavouring agent in an amount of about 0.2 to about 0.4% w/w in the tablet core.

In one embodiment, the dolutegravir sodium granules and extragranular components are blended and then compressed into a tablet.

Tabletting methods are well known in the art of pharmacy. Techniques and formulations generally are found in Remington’s Pharmaceutical Sciences (Mack Publishing Co., Easton, PA), which is hereby incorporated by reference herein in its entirety. A tablet can be made by compression or moulding. Compressed tablets may be prepared by compressing in a suitable machine the active ingredient and one or more excipients in a free-flowing form such as a powder or granules.

In another embodiment, the compressed tablet is film coated.

In one embodiment, the dispersible tablets of the invention are round with a diameter of 6 mm. In another embodiment of the invention the dispersible tablet of the invention are coated and weigh about 93 mg each.

In a third aspect, the present invention provides a dispersible tablet formulation comprising dolutegravir or a pharmaceutically acceptable salt thereof and a pharmaceutically acceptable ion for use in therapy.

In a fourth aspect, the present invention provides a dispersible tablet formulation comprising dolutegravir or a pharmaceutically acceptable salt thereof and a pharmaceutically acceptable ion for use in the treatment of HIV infection, in particular HIV infection in a pediatric patient.

In a fifth aspect, the present invention provides a method of treating HIV infection, for example HIV infection in a pediatric patient, which method comprises administering to said patient a dispersible tablet formulation comprising dolutegravir or a pharmaceutically acceptable salt thereof and a pharmaceutically acceptable ion.

The dispersible tablet formulations of the invention are typically administered once per day.

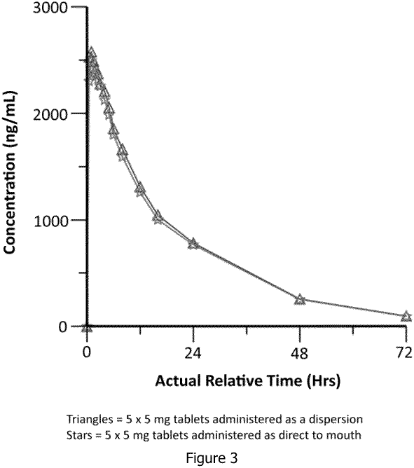

The dispersible tablet formulaitons of the invention can be dispersed in water prior to administration or administered direct to mouth (see FIG. 3).

The dispersible tablet formulations of the invention are typically administered indefinitely to maintain the desired therapeutic effect. It will be further understood by such skilled artisans that suitable dosing regimens may require adjustment for an individual patient.

The dispersible tablet formulations of the invention typically comprise 5 mg of dolutegravir in free acid form. As the skilled person will appreciate, the number of tablets required per dose may depend on the size of the patient. In one embodiment, for children weighing from 3 kg to less than 6 kg one tablet per day is dosed. In another embodiment, for children weighing from 6 kg to less than 10 kg 3 tablets per day are dosed. In another embodiment, for children weighing from 10 kg to less than 14 kg 4 tablets per day are dosed. In another embodiment, for children weighing from 14 kg to less than 20 kg 5 tablets per day are dosed. In a further embodiment, for children weighing 20 kg and greater 6 tablets per day are dosed.

In a sixth aspect, the present invention provides a kit comprising a dispersible tablet formulation comprising dolutegravir or a pharmaceutically acceptable salt thereof and a pharmaceutically acceptable ion, together with instructions for the use thereof for the treatment of HIV infection.

In one embodiment, the kit comprises 60 tablets, for example 60 tablets in a 60 cc bottle with a child-resistant closure optionally with 2 g of desiccant. In another embodiment, the kit comprises a suitable dosing system, including but not limited to a dosing cup and syringe. In a further embodiment, the kit comprises instructions regarding the amount of water in which to disperse the tablet(s). For example, for a dose of from 1 to 3 tablets, the tablets are typically dispersed in about 5 ml of water. For a dose of from 4 to 6 tablets, the tablets are typically dispersed in about 10 ml of water.

In a seventh aspect, the present invention provides a combination of a dispersible tablet formulation comprising dolutegravir or a pharmaceutically acceptable salt thereof and a pharmaceutically acceptable ion, together with one or more other therapeutic agents.

In one embodiment, the other therapeutic agent is a nucleoside reverse transcriptase inhibitor such as abacavir, didanosine, emtricitabine, lamivudine, stavudine, tenorfovir alafenamide fumarate, tenofovir disoproxil fumarate or zidovudine; a non-nucleoside reverse transcriptase inhibitor such as delaviridine, doravine, efavirenz, etravirine, nevirapine or rilpivirine; a protease inhibitor such as atazanavir, darunavir, fosamprenavir, indinavir, lopinavir, nelfinavir, saquinavir or tipranavir; a fusion inhibitor such as enfuvirtide; a CCR5 antagonist such as maraviroc; a cytochrome P4503A inhibitor such as ritonavir or cobicistat; an integrase inhibitor such as dolutegravir, raltegravir, elvitegravir, bictegravir or cabotegravir; or a post attachment-inhibitor such as ibalizumab-uiyk.

The individual components of such combinations may be administered either sequentially or simultaneously in separate or combined formulations.

### EXAMPLES

### Dolutegravir Solubility in the Presence of Monovalent and Divalent Ions

Solubility was determined by dissolving 20 mg of dolutegravir into 12 ml of water containing the specified ion (see Table 1) at a concentration of 500 ppm.

TABLE 1

| Ion (500 ppm) | Dolutegravir Solubility (mg/ml) |
|---|---|
| Sodium | 0.46 |
| Potassium | 0.78 |
| Calcium | 0.03 |
| Magnesium | 0.21 |
| None (purified water) | 1.20 |

### Dolutearavir Sodium 5 mg Dispersible Tablet

TABLE 2

| Excipient | Function | Level (%w/w in granule) | Level (%w/w in tablet core) [and potential range for each level] |
|---|---|---|---|
| Intragranular Components | | | |
| 5 mg free acid equivalent of dolutegravir sodium | Active ingredient | | |
| Mannitol | Diluent | 50.5 | 16.2 [13.8 - 20.3] |
| Microcrystalline Cellulose | Processing Aid | 20.8 | 6.7 [5.7 - 8.4] |
| Povidone K29/32 | Binder | 5.2 | 1.7 [1.4 - 2.1] |
| Sodium Starch Glycolate (Type A) | Disintegrant | 5.2 | 1.7 [1.4 - 2.1] |
| Purified water¹ | Vehicle | q.s. | q.s. |
| Extragranular Components | | | |
| Silicified Microcrystalline Cellulose | Processing Aid | - | 53.3 [45.3 - 66.2] |
| Crospovidone (Type B) | Disintegrant | - | 10.0 [8.5 - 12.5] |
| Calcium Sulfate Dihydrate | Solubility Modifier | - | 2.2 [1.9 - 2.8] |
| Sucralose | Sweetener | - | 0.7 [0.6 - 0.8] |
| Strawberry Cream Flavour PHS-132963 | Flavour | - | 0.3 [0.3 - 0.4] |
| Sodium Stearyl Fumarate | Lubricant | - | 1.5 [1.3 - 1.9] |
| Film Coating | | | |
| Aquarious BP18237 White fim Coat or Opadry OY-S-28876 White Film Coat | Film Coat | | 2.0-4.0 [1.7 - 5] |
| Purified water | Vehicle | | q.s. |

¹ Purified water is removed during processing

### Specification for Dolutearavir Sodium 5 mg Dispersible Tablet

TABLE 3

| Test | Acceptance Criteria |
|---|---|
| Description | A white, round, biconvex tablet, with “SV H7S” debossed on one face and “5” debossed on the other face |
| Identification of dolutegravir | |
| by HPLC¹ | The retention time of the principal peak in the sample chromatogram corresponds with that of the principal peak in the dolutegravir reference material chromatogram |
| by UV¹ | The absorbance maxima in the spectrum of the sample correspond to the absorbance maxima in the reference standard |
| Dolutegravir content by HPLC (% label claim)² | 90.0 - 110.0 |
| Uniformity of Dosage Units by HPLC¹ | Complies with Pharmacopoeia (USP <905>) |
| Dolutegravir drug-related impurities content by HPLC (% area)^{3,4,5} | |
| Any unspecified dolutegravir related degradation product | Not greater than 0.2 |
| Total dolutegravir degradation products | Not greater than 1.0 |
| Fineness of dispersion⁶ | Complies with Pharmacopoeia (Ph.Eur 0478) |
| Water content by Karl Fischer (%)⁵ | Not greater than 7.0 |
| Dissolution by UV (% dolutegravir released)⁷ | Complies with Pharmacopoeia (USP <711>) where Q = 80% at 25 minutes |
| Disintegration (minutes) | ≤3 |
| Microbiological Quality of Drug Product | Less than 0.60 |
| Water Activity (a_{w})^{1,8} (Tier 1) | |
| Microbial LimitsTest^{1,8} (Tier 2) | Complies with Harmonised Pharmacopeia * |
| Total Aerobic Microbial Count (TAMC) (CFU/g) | Not greater than 10³ |
| Total combined yeasts / mould count (TYMC) (CFU/g) | Not greater than 10² |
| Specified micro-organisms: Escherichia coli | Absent in 1 g |

Notes:
* Ph.Eur. / USP / JP
1. Performed at release only.
2. For batch release the mean result from the uniformity of dosage units test can be applied.
3. Does not include any process/synthetic impurities.
4. All degradation products greater than or equal to 0.05% are reported.
5. Not tested at release. Performed only on stability.
6. Fineness of dispersion test will be performed on a minimum of two batches per year if manufactured.
7. Quantification of amount dissolved is determined by HPLC or UV (dissolution test).
8. If the Water Activity criterion is met then MLT (Tier 2) testing is not required. If Water Activity testing is not performed or the acceptance criterion for Water Activity is not achieved, then MLT (Tier 2) testing must be performed

### Taste Assessment of Dolutearavir Sodium 5 mg Dispersible Tablet

The palatability and acceptability of the dispersible tablet formulation was assessed by providing respondents with the tablet and asking them to rate the taste on a scale of “very good” to “very bad”. The overall assessment is shown in Table 4. The dispersible tablet formulation taste was acceptable to the majority of respondents and there were very few problems with preparation or administration.

TABLE 4

| Overall taste assessment | 72 |
|---|---|
| Number of Respondents | |
| Very Good | 15 (21%) |
| Good | 45 (63%) |
| Average | 10 (14%) |
| Bad | 1 (1%) |
| Very Bad | 1 (1%) |

1. A dispersible tablet formulation comprising dolutegravir or a pharmaceutically acceptable salt thereof and a pharmaceutically acceptable ion.

2. A dispersible tablet formulation according to claim 1, wherein the dolutegravir or a pharmaceutically acceptable salt thereof is dolutegravir sodium.

3. A dispersible tablet formulation according to claim 1, wherein the tablet comprises 5 mg of dolutegravir free acid equivalent.

4. A dispersible tablet formulation according to claim 1, wherein the a pharmaceutically acceptable ion is calcium.

5. A dispersible tablet formulation according to claim 4 wherein the calcium is present in an amount of up to 5% w/w in the tablet core.

6. A dispersible tablet formulation according to claim 1 comprising a flavouring agent.

7. A dispersible tablet formulation according to claim 7 wherein the flavouring agent is present in an amount of up to 2% w/w in the tablet core.

8. A dispersible tablet formulation according to claim 1 comprising sucralose.

9. A dispersible tablet formulation according to claim 8 wherein the sucralose is present in an amount of up to 2% w/w in the tablet core.

10. A dispersible tablet formulation according to claim 1, wherein the tablet comprises a coating.

11. A process for making a dispersible tablet formulation as claimed in claim 1 comprising mixing dolutegravir or a pharmaceutically acceptable salt thereof and a pharmaceutically acceptable ion.

12. A dispsersible tablet formulation as claimed in claim 1 for use in therapy.

13. A dispsersible tablet formulation as claimed in claim 1 for use in the treatment of HIV infection.

14. A method of treating HIV infection, which method comprises administering to said patient a dispersible tablet formulation as claimed in claim 1.
